(19) United States
(12) Patent Application Publication
(10) Pub. No.: US 2019/0002691 A1
(43) Pub. Date: Jan. 3, 2019

(54) COPOLYMER, PRODUCTION METHOD THEREOF, AND RESIN COMPOSITION

(71) Applicant: MITSUI CHEMICALS, INC., Tokyo (JP)

(72) Inventors: Ryohei OGAWA, Ichihara-shi, CHIBA (JP); Chojiro HIGUCHI, Yokohama-shi, KANAGAWA (JP); Tatsuhiro URAKAMI, Ichihara-shi, CHIBA (JP)

(21) Appl. No.: 15/780,318

(22) PCT Filed: Nov. 30, 2016
(86) PCT No.: PCT/JP2016/085524

§ 371 Date: May 31, 2018

(30) Foreign Application Priority Data
Dec. 4, 2015 (JP) 2015-237343

(51) Int. Cl. C08L 77/04; C08G 69/10; C08G 69/04; C08L 67/00; A61K 47/34

(52) U.S. Cl. CPC C08L 77/04; C08G 69/10; C08G 69/04; C08L 2201/06; A61K 47/34; C08G 2230/00; C08L 67/00

(57) ABSTRACT

Disclosed are a water-insoluble copolymer having a constitutional unit (X) derived from a hydroxycarboxylic acid and a constitutional unit (Y) derived from an amino group-containing polyvalent carboxylic acid, wherein the molar ratio (X/Y) of constitutional units is 2/1≤(X/Y)<8/1, and the amide bond proportion of the constitutional unit (Y) represented by the following formula (1) is defined by the following formulae (2-1) to (2-3); a production method thereof, and a resin composition containing the copolymer.

Amide bond proportion (%)=A/Asp×100 (1)

[A=number of moles of an amide bond in (Y), Asp=number of moles of (Y)]

[when 2/1≤(X/Y)<4/1] amide bond proportion (%)≥25 (2-1)

[when 4/1≤(X/Y)≤6.5/1] amide bond proportion (%)≥30 (2-2)

[when 6.5/1<(X/Y)<8/1] amide bond proportion (%)≥50 (2-3)

### TECHNICAL FIELD

The present invention relates to a copolymer which is useful in an application for promoting hydrolysis of other resins, a production method thereof, and a resin composition containing the copolymer.

### BACKGROUND ART

Conventionally, resins typified by, for example, polylactic acid, polyglycolic acid and polycaprolactone are utilized in various applications in the form of, for example, film and fiber as the biodegradable resin which is degraded by moisture or an enzyme under natural circumstances or intravitally.

For example, polylactic acid is used in applications of, for example, disposable vessels and packaging materials since polylactic acid shows good processability and a molded article of polylactic acid is excellent in mechanical strength. However, since the degradation speed of polylactic acid under conditions other than compost (for example, in seawater, in soil) is relatively slow, polylactic acid is not readily used in applications requiring degradation and disappearance in several months. When polylactic acid is used in an sustained release formulation, the degradation speed of polylactic acid in vivo is slow, thus, polylactic acid remains in the body for a long period of time after releasing of a drug. Hence, polylactic acid cannot sufficiently meet the need for a formulation which releases a drug slowly in a relatively short period of time.

That is, the degradability of biodegradable resins is not necessarily sufficient depending on applications. Therefore, there are recently investigations on additives for promoting hydrolysis to enhance degradation thereof. For such purpose, for example, Patent Document 1 discloses block or graft copolymers having a hydrophilic segment derived from a polyamino acid and a hydrophobic segment composed of a degradable polymer. Patent Document 2 discloses copolymers having a constitutional unit derived from a polyvalent carboxylic acid excluding an amino acid and a constitutional unit derived from a hydroxycarboxylic acid. Patent Document 3 discloses copolymers having a constitutional unit derived from a polyvalent carboxylic acid and a constitutional unit derived from a hydroxycarboxylic acid.

As copolymers of such type, further, Patent Document 4 discloses a copolymer having a succinimide unit and a hydroxycarboxylic acid unit together, Non-Patent Document 1 discloses a novel copolymer obtained from aspartic acid and a lactide, Non-Patent Document 2 discloses a novel method of synthesizing an aspartic acid-lactic acid copolymer by direct-melt-polycondensation, and Non-Patent Document 3 discloses a method of synthesizing a copolymer of aspartic acid with lactic acid or glycolic acid using a specific catalyst.

As a result of repeated studies by the present inventors, however, it has been found that any conventional copolymers have still room for improvement of the ability of promoting hydrolysis and preservation stability. For example, under specific polymerization conditions described in Patent Documents 1 and 4 and Non-Patent Documents 1 and 2, the block ratio of the molecular chain of a copolymer increases, and the hydrolysis promoting effect lowers correspondingly. In the copolymer described in Non-Patent Document 3, the amount of lactic acid or glycolic acid with respect to aspartic acid is small, and compatibility with a biodegradable resin correspondingly lowers. The copolymer described in Patent Document 2 has low glass transition temperature, thus, preservation stability thereof is problematic, since the copolymer is obtained by using polyvalent carboxylic acids (for example, malic acid and citric acid) excluding amino acids. The copolymer described in preparation examples of Patent Document 3 has problems, for example, that the glass transition temperature thereof is low because of low molecular weight, and preservation stability thereof is poor.

### RELATED ART DOCUMENTS

### Patent Documents

Patent Document 1: JP 2000-345033 A

Patent Document 2: WO2012/137681

Patent Document 3: WO2014/038608

Patent Document 4: JP 2000-159888 A

### Non-Patent Documents

Non-Patent Document 1: Hosei Shinoda et al., “Synthesis and Characterization of Amphiphilic Biodegradable Copolymer, Poly(aspartic acid-co-lactic acid)”, Macromol. Biosci. 2003, 3, pp. 34-43
Non-Patent Document 2: Rui-Rong Ye et al., “Synthesis of Biodegradable Material Poly(lactic acid-co-aspartic acid) via Direct Melt Polycondensation and Its Characterization”, J. Appl. Polym. Sci. 2011, 121, pp. 3662-3668
Non-Patent Document 3: Ganpat L. Jain et al., Synthesis and Characterization of Random Copolymers of Aspartic Acid with Lactic Acid and Glycolic Acid”, Macromol. Chem., 1981, 182, pp. 2557-2561

### SUMMARY OF THE INVENTION

### Technical Problem

The present invention has been made for solving the problems of conventional technologies as described above. That is, the present invention has an object of providing a copolymer excellent in preservation stability, having good compatibility with other resins (for example, biodegradable resins) and excellent in the ability of promoting hydrolysis of other resins; a production method thereof, and a resin composition containing the copolymer.

### Solution to Problem

The present invention is specified by the following items.

[1] A water-insoluble copolymer having a constitutional unit (X) derived from a hydroxycarboxylic acid and a constitutional unit (Y) derived from an amino group-containing polyvalent carboxylic acid, wherein

the molar ratio (X/Y) of the constitutional unit (X) to the constitutional unit (Y) is 2/1≤(X/Y)<8/1, and

the amide bond proportion of the constitutional unit (Y) represented by the following formula (1) is defined by the following formulae (2-1) to (2-3):

amide bond proportion (%)=A/Asp×100 (1)

(wherein, A is the number of moles of an amide bond in the constitutional unit (Y) calculated by the ¹H-NMR spectrum measured in deuterated dimethylformamide, and Asp is the number of moles of the constitutional unit (Y) in the copolymer.)
[when 2/1≤(X/Y)<4/1]

amide bond proportion (%)≤25 (2-1)

[when 4/1≤(X/Y)≤6.5/1]

amide bond proportion (%)≥30 (2-2)

[when 6.5/1<(X/Y)<8/1]

amide bond proportion (%)≥50 (2-3).

[2] The copolymer according to [1], wherein the weight-average molecular weight measured by size exclusion chromatography using dimethylacetamide as an eluent is 8000 or more and 50000 or less.

[3] The copolymer according to [1], wherein the inherent viscosity in dimethylacetamide is 0.05 dl/g or more and 0.20 dl/g or less.

[4] The copolymer according to [1], wherein the acid value is 0.2 mmol/g or more and 2.5 mmol/g or less.

[5] The copolymer according to [1], wherein the copolymer has a glass transition temperature of 40° C. or higher and is amorphous having substantially no melting point.

[6] A method for producing the copolymer of [1], comprising a step of polymerizing a hydroxycarboxylic acid and an amino group-containing polyvalent carboxylic acid by direct dehydration and condensation.

[7] The production method according to [6], wherein the polymerization is conducted at a reaction temperature of 170° C. or lower until the amino group-containing polyvalent carboxylic acid is dissolved.

[8] The production method according to [6], wherein the polymerization is conducted at a reaction pressure of 100 mmHg or less.

[9] The production method according to [6], wherein the polymerization is conducted using a catalyst.

[10] The production method according to [9], wherein the polymerization is conducted using one or two or more kinds of catalysts selected from the group consisting of tin, titanium, zinc, aluminum, calcium, magnesium and organic acids.

[11] A resin composition comprising the copolymer (A) of [1] and a resin (B) selected from the group consisting of polyolefin resins, polystyrene resins, polyester resins, polycarbonate resins and degradable resins, wherein the mass ratio (A/B) of the copolymer (A) to the resin (B) is 1/99 to 50/50.

[12] The resin composition according to [11], wherein the resin (B) is a degradable resin.

[13] The resin composition according to [12], wherein the degradable resin is an aliphatic polyester.

[14] The resin composition according to [11], wherein the reduced viscosity of the copolymer (A) in dimethylacetamide is 0.05 or more and 0.20 or less.

[15] A method for promoting hydrolysis of a resin (B) having a weight-average molecular weight of 3000 or more and 500000 or less selected from the group consisting of polyolefin resins, polystyrene resins, polyester resins, polycarbonate resins and degradable resins, wherein the copolymer (A) according to [1] is mixed with the resin (B) so that the mass ratio (A/B) of the copolymer (A) to the resin (B) is 1/99 to 50/50.

[16] The method according to [15], wherein the resin (B) is an aliphatic polyester.

### Effect of the Invention

According to the present invention, a copolymer excellent in preservation stability, having good compatibility with other resins (for example, biodegradable resins) and excellent in the ability of promoting hydrolysis of other resins is obtained.

### BRIEF EXPLANATION OF DRAWINGS

FIG. 1 is a graph showing a relation between the aspartic acid proportion and the amide bond proportion in each copolymer in examples and comparative examples.

FIG. 2 is a graph showing the results of the hydrolysis promoting test in examples and comparative examples.

### MODES FOR CARRYING OUT THE INVENTION

### <Copolymer (A)>

The copolymer (A) of the present invention is a water-insoluble copolymer having a constitutional unit (X) derived from a hydroxycarboxylic acid and a constitutional unit (Y) derived from an amino group-containing polyvalent carboxylic acid.

In the present invention, “water-insoluble” means that when a polymer is put into water at normal temperature (23° C.) and even if this is stirred sufficiently, the polymer is not substantially dissolved in water. Specifically, if no change is recognized by visual observation between condition of the polymer powder in water directly after input and condition of the polymer powder in water after sufficient stirring, those skilled in the art can easily judge that the polymer is “water-insoluble”. Patent Document 4 explained previously describes also a copolymer which is made water-soluble by hydrolyzing an imide ring in the copolymer to generate a carboxyl group, however, such a water-soluble copolymer has problems, for example, that preservation stability is poor because of low glass transition temperature, and the molecular weight lowers remarkably in kneading with other resins (for example, biodegradable resins). In contrast, the copolymer (A) of the present invention does not cause such problems since the copolymer (A) is water-insoluble.

In the copolymer (A) of the present invention, the molar ratio (X/Y) of a constitutional unit (X) derived from a hydroxycarboxylic acid to a constitutional unit (Y) derived from an amino group-containing polyvalent carboxylic acid is 2/1≤(X/Y)<8/1, and the amide bond proportion of the constitutional unit (Y) represented by the following formula (1) is defined by the following formulae (2-1) to (2-3).

Amide bond proportion (%)=A/Asp×100 (1)

(wherein, A is the number of moles of an amide bond in the constitutional unit (Y) calculated by the ¹H-NMR spectrum measured in deuterated dimethylformamide, and Asp is the number of moles of the constitutional unit (Y) in the copolymer.)
[when 2/1≤(X/Y)<4/1]

amide bond proportion (%)≥25 (2-1)

[when 4/1≤(X/Y)≤6.5/1]

amide bond proportion (%)≥30 (2-2)

[when 6.5/1<(X/Y)<8/1]

amide bond proportion (%)≥50 (2-3)

This amide bond proportion (%) is a value calculated from the ¹H-NMR spectrum obtained by using a nuclear magnetic resonance apparatus.

The amide bond proportion is an index for the amount of a long chain branched structure in the copolymer (A). For example, the high amide bond proportion means that there are a lot of positions at which a constitutional unit (X) derived from a hydroxycarboxylic acid and a constitutional unit (Y) derived from an amino group-containing polyvalent carboxylic acid are amide-bonded directly in the copolymer (A). At the amide bond portion, a branched structure is necessarily generated, and a carboxyl group is present at the end of its branched structure. That is, when an alternating property of the constitutional unit (X) and the constitutional unit (Y) in the molecular chain is high (block ratio is low), the number of branched structures increases, and accordingly, a larger number of carboxyl groups are present at the molecular chain end.

Therefore, when the amide bond proportion is higher, a larger number of carboxyl groups are present at the molecular chain end of the copolymer (A), and the ability of promoting hydrolysis of other resins improves.

Further, when the amide bond proportion is higher, an alternating property of the constitutional unit (X) and the constitutional unit (Y) increases (block ratio is lowered), thus, compatibility with other resins (for example, biodegradable resins) increases as compared with conventional copolymers having high block ratio, and as a result, the ability of promoting hydrolysis is improved.

When the amide bond proportion is higher, the glass transition temperature of a copolymer increases because of a hydrogen bond between molecules, and preservation stability (for example, anti-blocking property) at a place undergoing high temperature such as a warehouse improves. This effect is effective particularly in the case of the above-described formula (2-2) [4/1≤(X/Y)≤6.5/1]. The reason for this is that since the copolymer (A) having such molar ratio (X/Y) tends to have low original glass transition temperature, it is highly necessary to raise the glass transition temperature by the action of a hydrogen bond.

The constitutional unit (X) may advantageously be a constitutional unit derived from a hydroxycarboxylic acid and is not particularly restricted. The valence of a hydroxycarboxylic acid (number of hydroxyl group) is preferably 1 to 4, more preferably 1 to 2, most preferably 1. Particularly, constitutional units derived from α-hydroxycarboxylic acids such as lactic acid, glycolic acid, 2-hydroxybutyric acid, 2-hydroxyvaleric acid, 2-hydroxycaproic acid and 2-hydroxycapric acid; lactide, glycolide, p-dioxanone, β-propiolactone, β-butyrolactone, δ-valerolactone or ε-caprolactone are preferable, and constitutional units derived from lactic acid or lactide are more preferable. These constitutional units (X) may be contained each singly or two or more of them may be contained. For example, lactide is a cyclic dimer of lactic acid and glycolide is a cyclic dimer of glycolic acid, and they are ring-opened in polymerization and react as a hydroxycarboxylic acid. Therefore, constitutional units using these cyclic dimers as the raw material are also included as the constitutional unit derived from a hydroxycarboxylic acid.

The constitutional unit (Y) may advantageously be a constitutional unit derived from an amino group-containing polyvalent carboxylic acid and is not particularly restricted. The valence of the amino group-containing polyvalent carboxylic acid (number of carboxyl group) is preferably 2 to 4, more preferably 2 to 3, most preferably 2. Particularly, constitutional units derived from aspartic acid, glutamic acid or aminodicarboxylic acid are preferable. The constitutional unit (Y) may form a cyclic structure such as an imide ring, and the cyclic structure may be ring-opened, or these may be mixed. These constitutional units (Y) may be contained each singly or two or more of them may be contained.

In the copolymer (A), constitutional units other than the constitutional unit (X) and the constitutional unit (Y) may be present. It is necessary that the amount thereof is such that the nature of the copolymer (A) is not impaired significantly. From such standpoint, the amount is desirably 0 to 20% by mole with respect to 100% by mole of all constitutional units of the copolymer (A).

The weight-average molecular weight (Mw) of the copolymer (A) of the present invention is preferably 8000 to 50000 g/mol, more preferably 10000 to 30000 g/mol, particularly preferably 12000 to 25000 g/mol. This Mw is a value measured using standard polystyrene by size exclusion chromatography (SEC) using dimethylacetamide as an eluent described later. It is well known that the weight-average molecular weight obtained by SEC varies significantly depending on conditions such as differences in, for example, the eluent, the column and the standard sample for relative comparison to be used. The weight-average molecular weight of the copolymer (A) of the present invention is a measured value when dimethylacetamide is used as an eluent under conditions shown in examples described later. Meanwhile, for example, Patent Document 3 discloses a measured value when chloroform is used as an eluent. For making comparison with the present invention easy, the weight-average molecular weight of a specific copolymer when chloroform was used as an eluent was also measured in examples described later, and correlative relationship between both measured values was examined.

The inherent viscosity of the copolymer (A) of the present invention in dimethylacetamide is preferably 0.05 dl/g or more and 0.20 dl/g or less, more preferably 0.08 dl/g or more and 0.15 dl/g or less. This inherent viscosity is a value measured by a Ubbelohde viscometer tube using a prepared dimethylacetamide solution of a sample of specific concentration.

The acid value of the copolymer (A) of the present invention is preferably 0.2 mmol/g or more and 2.5 mmol/g or less, more preferably 0.8 mmol/g or more and 2.0 mmol/g or less. This acid value is a value measured by a potentiometric titrator using a solution prepared by dissolving about 0.5 g of a sample in 30 mL of a mixed solution of chloroform/methanol (volume ratio: 70/30). As describe previously, when the amide bond proportion is high, the number of branched structures increases, and accordingly, a larger number of carboxyl groups are present at the molecular chain end. As a result, the acid value of the copolymer (A) becomes relatively higher. When the acid value becomes higher, degradation promoting ability when mixed with other resins improves. For general linear polymers, when the molecular weight becomes higher (when degree of polymerization is enhanced), the acid value becomes smaller. In contrast, for the copolymer (A) of the present invention, it is possible to raise the molecular weight and simultaneously to increase also the acid value, by increasing the number of branched structures.

The glass transition temperature of the copolymer (A) of the present invention is preferably 40° C. or higher, more preferably 52° C. to 120° C., particularly preferably 55° C. to 70° C., and it is preferable that the copolymer (A) is amorphous having substantially no melting point. This glass transition temperature and the melting point are values measured by DSC. As described previously, when the amide bond proportion in the copolymer (A) of the present invention increases, the glass transition temperature also increases, and resultantly, preservation stability (for example, anti-blocking property) improves. When the copolymer is amorphous, there is no need to melt it at high temperature. To increase the glass transition temperature is effective particularly when the number of structures essentially tending to increase the glass transition temperature such as a succinimide block structure is small in the copolymer (A). “Having substantially no melting point” means specifically that melting point is not observed when DSC measurement is conducted under conditions in examples described later.

The production method of the copolymer (A) of the present invention is not particularly restricted. It can be obtained, for example, by mixing a hydroxycarboxylic acid and an amino group-containing polyvalent carboxylic acid, and subjecting them to direct dehydration and condensation under reduced pressure with heating in the presence or absence of a catalyst.

For obtaining a copolymer like the copolymer (A) of the present invention, in which an alternating property of the constitutional unit (X) and the constitutional unit (Y) is high (block ratio is low) and the number of branched structures is large, particularly it is preferable that the reaction temperature is set at lower temperature than in conventional methods until the amino group-containing polyvalent carboxylic acid is dissolved. Specifically, its reaction temperature is preferably 17° C. or lower, more preferably 140° C. to 160° C. For obtaining a copolymer like the copolymer (A) of the present invention, in which the amide bond proportion is high, it is important to conduct polymerization in view of reactivity (for example, reaction speed) of each functional group. According to knowledge of the present inventors, it has been found that a copolymer in which an alternating property is high (block ratio is low) and the number of branched structures is large tends to be obtained easily, for example, by suppressing the reaction speed of a specific functional group of the amino group-containing polyvalent carboxylic acid by setting the reaction temperature at relatively lower temperature until the amino group-containing polyvalent carboxylic acid is dissolved. Even if the reaction temperature is set at 170° C. or lower, the copolymer (A) of the present invention is not necessarily obtained, and it is preferable to appropriately consider other various conditions in the reaction such as the dehydration speed of byproduct water generated by the reaction, and the stirring conditions. The specific method for quickly dehydrating by-product water includes, for example, use of a reactor increasing the contact area of the reaction liquid with a gaseous layer part, speeding up of stirring rate, use of a stirring blade of high stirring efficiency such as a max blend blade, blowing of an inert gas into the reaction system, and use of an azeotropic solvent. After the amino group-containing polyvalent carboxylic acid is dissolved completely and the dehydration reaction progresses sufficiently, it may be heated at high temperature over 170° C. The reason for this is guessed that when the carboxylic acid is dissolved completely, an amide bond is formed sufficiently by the reaction of the amino group-containing polyvalent carboxylic acid and a hydroxycarboxylic acid, and the hydrolysis reaction of the generated amide bond is suppressed.

It is preferable that the polymerization step for production of the copolymer (A) of the present invention is conducted under reduced pressure by stages for the purpose of efficiently removing water generated with the progress of the polymerization reaction. The pressure is preferably 100 mmHg or less, more preferably 100 to 10 mmHg. It is also preferable to further reduce the pressure by stages with the progress of polymerization. Under such polymerization conditions, a copolymer having a lot of branched structures and having high molecular weight tends to be obtained. The reaction time is preferably 10 to 40 hours, more preferably 15 to 30 hours.

In the polymerization step for production of the copolymer (A) of the present invention, use of a catalyst is preferable since the reaction speed is increased, namely, the copolymer (A) can be produced in a relatively short period of time. The catalyst includes, for example, one or two or more kinds of catalysts selected from the group consisting of tin, titanium, zinc, aluminum, calcium, magnesium and organic acids. Of them, divalent tin, titanium and organic acids are preferable.

Though the application of the copolymer (A) of the present invention described above is not particularly restricted, it is preferable to use the copolymer (A) for promoting hydrolysis of other resins. The kind of the other resin is not particularly restricted provided that the effect by the copolymer (A) of the present invention is obtained.

### <Resin (B)>

The resin (B) is a resin selected from the group consisting of polyolefin resins, polystyrene resins, polyester resins, polycarbonate resins and degradable resins. It is particularly effective to use the copolymer (A) of the present invention for promoting hydrolysis of this resin (B).

Specific examples of the polyolefin resins include, for example, homopolymers or copolymers synthesized from one or more olefin monomers such as ethylene, propylene and butylene such as high density polyethylene, low density polyethylene, linear low density polyethylene, polypropylene, polyisopropylene, polyisobutylene and polybutadiene, copolymers with any other monomers, or mixtures thereof.

Specific examples of the polystyrene resins include, for example, polystyrene, acrylonitrile-butadiene-styrene copolymer, homopolymers or copolymers synthesized from one or more styrene monomers, copolymers with any other monomers, or mixtures thereof.

Specific examples of the polyester resins include (1) polyhydroxycarboxylic acids such as homopolymers or copolymers synthesized from one or more hydroxycarboxylic acids such as α-hydroxy monocarboxylic acids (for example, glycolic acid, lactic acid, 2-hydroxybutyric acid, 2-hydroxyvaleric acid, 2-hydroxycaproic acid, 2-hydroxycapric acid), hydroxy dicarboxylic acids (for example, malic acid), and hydroxy tricarboxylic acids (for example, citric acid), copolymers with any other monomers, or mixtures thereof; (2) polylactides such as homopolymers or copolymers synthesized from one or more lactides such as glycolide, lactide, benzylmalolactonate, malite benzyl ester, and 3-[(benzyloxycarbonyl)methyl]-1,4-dioxane-2,5-dione, copolymers with any other monomers, or mixtures thereof; (3) polylactones such as homopolymers or copolymers synthesized from one or more lactones such as β-propiolactone, δ-valerolactone, ε-caprolactone, and N-benzyloxycarbonyl-L-serine-β-lactone, copolymers with any other monomers, or mixtures thereof. Particularly, these can be copolymerized also with, for example, glycolide, and lactide as a cyclic dimer of an α-hydroxy acid.

Specific examples of the polycarbonate resins include homopolymer or copolymers synthesized from one or more monomers such as polyoxymethylene, polybutylene terephthalate, polyethylene terephthalate and polyphenylene oxide, homopolymers or copolymers synthesized from copolymers with any other monomers, copolymers with any other monomers, or mixtures thereof.

The degradable resin includes polyester resins (1) to (3) listed above, and polyanhydrides such as poly[1,3-bis(p-carboxyphenoxy)methane] and poly(terephthalic acid-sebacic acid anhydride); degradable polycarbonates such as poly(oxycarbonyloxyethylene) and spiroorthopolycarbonate; poly-ortho esters such as poly{3,9-bis(ethylidene-2,4,8,10-tetraoxaspiro[5,5]undecane-1,6-hexanediol}; poly-α-cyanoacrylates such as poly-a-cyanoacryilc acid isobutyl; polyphosphazenes such as polydiamino-phosphazene; other degradable resins such as microbial synthetic resins typified by, for example, polyhydroxy esters, and resins obtained by blending, for example, starch, modified starch, hide powder or micronized cellulose into the above-described various resins.

Of various resins listed above, polyolefin resins, polycarbonate resins and degradable resins are preferable, and particularly, degradable resins are preferable, since the copolymer (A) and the resin (B) are mixed more uniformly without separation. Of degradable resins, aliphatic polyesters, polylactides and polylactones are preferable, aliphatic polyesters are more preferable, polyhydroxycarboxylic acids (for example, polylactic acid, lactic acid-glycolic acid copolymer, polycaprolactone) are most preferable, from the standpoint of compatibility with the copolymer (A).

In the present invention, the molecular weight of the resin (B) is not particularly restricted. The weight-average molecular weight of the resin (B) is preferably 3000 or more and 500000 or less, more preferably 10000 or more and 300000 or less, in view of easiness of mixing with the copolymer (A).

### <Resin Composition>

The resin composition of the present invention is a composition containing the copolymer (A) of the present invention and the resin (B) explained above. The resin composition of the present invention is suitable as a biodegradable resin composition which is degraded by moisture or an enzyme under natural circumstances or intravitally since the copolymer (A) suitably promotes hydrolysis of the resin (B) as described above.

In the resin composition of the present invention, the mass ratio (A/B) of the copolymer (A) to the resin (B) is 1/99 to 50/50, preferably 5/95 to 50/50.

The reduced viscosity of the copolymer (A) in the resin composition of the present invention in dimethylacetamide is preferably 0.05 or more and 0.20 or less, more preferably 0.08 or more and 0.15 or less.

### <Hydrolysis Promoting Method>

The hydrolysis promoting method of the present invention is a method of promoting hydrolysis of a resin (B) having a weight-average molecular weight of 3000 or more and 500000 or less by mixing a copolymer (A) with the resin (B) so that the mass ratio (A/B) of the copolymer (A) to the resin (B) is 1/99 to 50/50. This method is the production method of the resin composition of the present invention explained above, and simultaneously is a method particularly focusing on promotion of hydrolysis. Also in this context, the resin (B) is preferably an aliphatic polyester.

### EXAMPLES

The present invention will be illustrated specifically based on examples below, but the present invention is not limited to these examples. The measurement methods of physical properties are as described below.

### [Amide Bond Proportion of Constitutional Unit (Y)]

A copolymer was dissolved completely in deuterated dimethyl sulfoxide at room temperature so that its concentration was 5% (w/v), and the ¹H-NMR spectrum was measured using a 270 MHz nuclear magnetic resonance apparatus manufactured by JEOL. The amide bond proportion in the copolymer was calculated according to the following formula from the resultant spectrum. Integrated intensities are calculated in the following ranges when TMS is 0 ppm.

Ia: 9.23 to 7.75 ppm

Ib: 5.92 to 3.84 ppm

Ic: 4.38 to 4.08 ppm

Id: 2.04 to 0.28 ppm

Attributions of respective intensity ratios are shown below.

Ia: proton derived from amide

Ib: sum of methine derived from lactic acid and aspartic acid and proton derived from terminal hydroxyl group in lactic acid

Ic: methine proton derived from lactic acid end (intensity is equivalent to terminal hydroxyl group in lactic acid)

Id: methyl group derived from lactic acid

The amide bond proportion is calculated by the following formula using these intensity ratios.

Amide bond proportion (%)=[Ia/{Ib−(Id/3+Ic)}]×100

### [Measurement of Molecular Weight]

The weight-average molecular weight (Mw) and the number-average molecular weight (Mn) of a copolymer were calculated as the relative value of the three-dimensional standard curve made using standard polystyrene (molecular weight: 63000, 186000, 65500, 28500, 13000, 3790, 1270) using size exclusion chromatography (SEC) and using dimethylacetamide (DMAc) dissolving 5 mM lithium bromide and phosphoric acid as an eluent. The measurement conditions are shown below.

detector RID-10A manufactured by Shimadzu Corp.

column: PLgel 5 μm Mixed-C (2 columns) manufactured by Agilent Technologies

column temperature: 40° C.

flow rate: 1.0 mL/min

sample concentration: 20 mg/mL (injection amount: 100 μL)

The correlative relationship between Mw measured by SEC using DMAc as an eluent as described above and Mw measured by SEC using chloroform as an eluent as described in Patent Document 3 was examined for reference. Specifically, Mw values according to both methods of a copolymer obtained under the same conditions as in Example 1 and Comparative Example 1 described later were measured. The results are shown in Table 1.

TABLE 1

| Mw in the case of | Mw in the case of |
|---|---|
| DMAc eluent | chloroform eluent |
| 5200 | 1200 |
| 5600 | 2200 |
| 10000 | 5300 |
| 10600 | 7000 |
| 12700 | 8900 |
| 17900 | 13300 |

The correlative relationship between both measured values shown in Table 1 is believed to be represented by the following formula (i).

[Mw in the case of chloroform eluent]=0.9413×[Mw in the case of DMAc eluent]−3410 (i)

### [Inherent Viscosity]

A dimethylacetamide solution having a sample concentration of 4% was prepared, and the inherent viscosity (dl/g) was measured using a Ubbelohde viscometer tube.

The correlative relationship between the above-described inherent viscosity and Mw measured by SEC using DMAc as an eluent is represented by the following formula (ii).

[Mw]=261×10³×[inherent viscosity]−10400 (ii)

### [Acid Value]

About 0.5 g of a copolymer sample was weighed and dissolved in 30 mL of a mixed solution of chloroform/methanol (volume ratio: 70/30), and the acid value was calculated by an automatic potentiometric titrator (AT-510) manufactured by Kyoto Electronics Manufacturing Co., Ltd. using 0.1 N potassium hydroxide (2-propanol solution) as the titration liquid.

### [Glass Transition Temperature (Tg) and Melting Point]

Using DSC-50 manufactured by Shimadzu Corp., a copolymer sample weighed in an aluminum pan was heated from room temperature up to 150° C. at a temperature rising rate of 10° C./min under nitrogen flow, then, quenched down to 0° C., and again, heated up to 150° C. at a temperature rising rate of 10° C./min, and the glass transition temperature (intermediate point) and the melting point during this process were measured.

### Example 1

Into a 300 mL separable flask equipped with a stirring blade, a thermometer, a nitrogen introduction tube and a Dean-Stark trap having an attached condenser were charged 100.11 g of 90% L-lactic acid (HP-90) manufactured by Purac and 26.62 g of aspartic acid manufactured by Wako Pure Chemical Industries, Ltd. This molar ratio of lactic acid to aspartic acid is 5/1. Further, tin chloride 2-hydrate was added so that the tin concentration was 2000 ppm, and the atmosphere in the flask was purged with nitrogen. The flask was immersed in an oil bath heated at 165° C., and the reaction mixture was dehydrated under nitrogen flow for 4 hours. The nitrogen flow was stopped, and the reaction mixture was stirred with heating at an internal temperature of 160° C. and at a degree of depressurization increased gradually like 100 mmHg for 5 hours, then, 30 mmHg for 10 hours followed by 10 mmHg for 2 hours, to obtain a copolymer.

### Example 2

A copolymer was obtained in the same manner as in Example 1, except that 300.33 g of 90% L-lactic acid (HP-90) manufactured by Purac and 79.86 g of aspartic acid manufactured by Wako Pure Chemical Industries, Ltd. (molar ratio: 5/1) were used.

### Example 3

A copolymer was obtained in the same manner as in Example 2, except that tin chloride 2-hydrate was not used.

### Example 4

Into a 500 mL 4-necked flask equipped with a stirring blade, a thermometer, a nitrogen introduction tube and a Dean-Stark trap having an attached condenser were charged 167 g of 90% L-lactic acid (HP-90) manufactured by Purac and 45 g aspartic acid manufactured by Wako Pure Chemical Industries, Ltd. This molar ratio of lactic acid to aspartic acid is 5/1. Further, tin chloride 2-hydrate was added so that the tin concentration was 2000 ppm, and the atmosphere in the flask was purged with nitrogen. The flask was immersed in an oil bath heated at 145° C., and the reaction mixture was dehydrated under nitrogen flow for 13 hours. The nitrogen flow was stopped, and the reaction mixture was stirred with heating at an internal temperature of 140° C. and at a degree of depressurization increased gradually like 100 mmHg for 5 hours, then, 30 mmHg for 11 hours followed by 10 mmHg for 12 hours, to obtain a copolymer.

### Example 5

A copolymer was obtained in the same manner as in Example 1, except that the molar ratio of lactic acid to aspartic acid was changed to 2/1.

### Example 6

A copolymer was obtained in the same manner as in Example 1, except that the molar ratio of lactic acid to aspartic acid was changed to 7.5/1.

### Example 7

Into a 500 mL separable flask equipped with a stirring blade, a thermometer, a nitrogen introduction tube and a Dean-Stark trap having an attached condenser were charged 300.33 g of 90% L-lactic acid (HP-90) manufactured by Purac and 79.86 g of aspartic acid manufactured by Wako Pure Chemical Industries, Ltd. This molar ratio of lactic acid to aspartic acid is 5/1. Further, 1.9 g of tin octanoate was added, and the atmosphere in the flask was purged with nitrogen. Under nitrogen flow, the flask was immersed in an oil bath, and heated up to 160° C. over a period of 1.5 hours, and the reaction mixture was further dehydrated for 3 hours at a stirring rate of 300 rpm, to attain complete dissolution of aspartic acid. Further, dehydration was continued for 1 hour under nitrogen flow. The dehydration amount at this time was 88 g. Thereafter, the nitrogen flow was stopped, and the reaction mixture was stirred with heating at an internal temperature of 160° C. and at a degree of depressurization increased gradually like 100 mmHg for 5 hours, then, 30 mmHg for 10 hours followed by 10 mmHg for 2 hours, to obtain a copolymer.

### Example 8

In the same manner as in Example 7, 300.33 g of lactic acid and 79.86 g of aspartic acid (molar ratio: 5/1) were charged into a separable flask, and 1.9 g of tin octanoate was added, and the atmosphere in the flask was purged with nitrogen. Then, under nitrogen flow, the flask was immersed in an oil bath, and heated up to 150° C. over a period of 1.5 hours, and the reaction mixture was further dehydrated for 3 hours at a stirring rate of 100 rpm, to attain complete dissolution of aspartic acid. Further, dehydration was continued for 3 hours under nitrogen flow. The dehydration amount at this time was 59 g. Thereafter, the nitrogen flow was stopped, and the reaction mixture was stirred with heating while gradually increasing a degree of depressurization under the same conditions as in Example 7, to obtain a copolymer.

### Example 9

Into a 2 L separable flask equipped with a stirring blade, a thermometer, a nitrogen introduction tube and a Dean-Stark trap having an attached condenser were charged 1802 g of 90% L-lactic acid (HP-90) manufactured by Purac and 479 g of aspartic acid manufactured by Wako Pure Chemical Industries, Ltd. This molar ratio of lactic acid to aspartic acid is 5/1. Further, 11.4 g of tin octanoate was added, and the atmosphere in the flask was purged with nitrogen. Under nitrogen flow, the flask was immersed in an oil bath, heated up to 150° C. over a period of 1.8 hours, and the reaction mixture was further dehydrated for 5 hours at a stirring rate of 300 rpm, to attain complete dissolution of aspartic acid. Further, dehydration was continued for 1 hour under nitrogen flow. The dehydration amount at this time was 390 g. Thereafter, the nitrogen flow was stopped, and the pressure was gradually reduced and kept at 100 mmHg for 3 hours. The integrated dehydration amount at this time was 567 g. Thereafter, the reaction mixture was heated up to 160° C., and stirred with heating at a degree of depressurization increased gradually like 30 mmHg for 10 hours followed by 10 mmHg for 4 hours, to obtain a copolymer.

### Example 10

In the same manner as in Example 9, 1802 g of lactic acid and 479 g of aspartic acid (molar ratio: 5/1) were charged into a separable flask, and 11.4 g of tin octanoate was added, and the atmosphere in the flask was purged with nitrogen. Then, under nitrogen flow, the flask was immersed in an oil bath, and heated up to 150° C. over a period of 2.5 hours, and the reaction mixture was further dehydrated for 5 hours at a stirring rate of 100 rpm, to attain complete dissolution of aspartic acid. Further, dehydration was continued for 1 hour under nitrogen flow. Thereafter, the nitrogen flow was stopped, and the pressure was reduced gradually and kept at 100 mmHg for 3 hours. The integrated dehydration amount at this time was 543 g. Thereafter, the reaction mixture was heated up to 180° C., and stirred with heating at a degree of depressurization of 30 mmHg for 10 hours, to obtain a copolymer. That is, the reaction was conducted at low temperature until aspartic acid was dissolved, and thereafter, polycondensation was conducted at high temperature.

### Comparative Example 1

Into a 300 mL separable flask equipped with a stirring blade, a thermometer, a nitrogen introduction tube and a Dean-Stark trap having an attached condenser were charged 72.1 g of L-lactide manufactured by Purac and 26.62 g of aspartic acid manufactured by Wako Pure Chemical Industries, Ltd. This molar ratio of lactic acid (converted from L-lactide) to aspartic acid is 5/1. The flask was immersed in an oil bath heated at 185° C., and aspartic acid was dissolved for 8 hours under nitrogen flow. Then, the flask was cooled until the inner temperature reached 130° C., then, tin octanoate was added so that the tin concentration was 2000 ppm, and the reaction mixture was stirred with heating under nitrogen flow at an internal temperature of 180° C. and at normal pressured for 25 hours, to obtain a copolymer.

### Comparative Example 2

A copolymer was obtained in the same manner as in Example 3, except that the reaction temperature was changed to 180° C.

### Comparative Example 3

A copolymer was obtained in the same manner as in Example 3, except that a 1500 mL separable flask was used, 1200 g of 90% L-lactic acid (HP-90) manufactured by Purac and 319.44 g of aspartic acid manufactured by Wako Pure Chemical Industries, Ltd. (molar ratio: 5/1) were used, and the reaction temperature (internal temperature) was changed to 180° C.

### Comparative Example 4

A copolymer was obtained in the same manner as in Comparative Example 1, except that the molar ratio of lactic acid to aspartic acid was changed to 2/1.

### Comparative Example 5

A copolymer was obtained in the same manner as in Comparative Example 1, except that the molar ratio of lactic acid to aspartic acid was changed to 7.5/1.

### Comparative Example 6

A copolymer was obtained in the same manner as in Comparative Example 1, except that the molar ratio of lactic acid to aspartic acid was changed to 10/1.

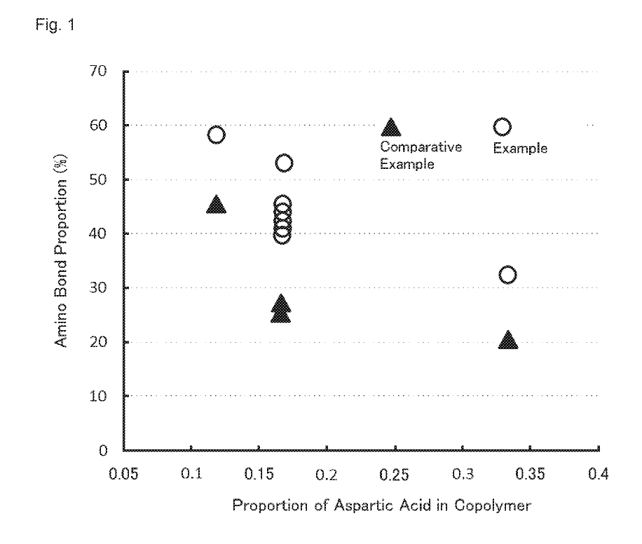

The analysis results of copolymers in examples and comparative examples described above are shown in Table 2. The relations between the aspartic acid proportion and the amide bond proportion in copolymers in examples and comparative examples are graphed in FIG. 1.

TABLE 2

| | Lactic | | | | | Proportion | | |
|---|---|---|---|---|---|---|---|---|
| | acid/ | Proportion | Reaction | | Inherent | of | Acid | |
| | aspartic | of | temperature | Mw | viscosity | amide | value | Tg |
| | acid | asparticcid | ° C. | g/mol | dL/g | bond | mmol/g | ° C. |
| Ex. 1 | 5/1 | 0.167 | 160 | 19400 | 0.110 | 45% | 1.36 | 62 |
| Ex. 2 | 5/1 | 0.167 | 160 | 15300 | 0.099 | 41% | 1.29 | 59 |
| Ex. 3 | 5/1 | 0.167 | 160 | 13300 | 0.090 | 41% | 1.40 | 58 |
| Ex. 4 | 5/1 | 0.167 | 140 | 12800 | 0.087 | 53% | 1.73 | 60 |
| Ex. 5 | 2/1 | 0.333 | 160 | 12800 | 0.086 | 32% | 2.11 | 82 |
| Ex. 6 | 7.5/1 | 0.118 | 160 | 24000 | 0.132 | 58% | 1.12 | 68 |
| Comp | 5/1 | 0.167 | 180 | 18600 | 0.111 | 25% | 0.90 | 57 |
| Ex. 1 | | | | | | | | |
| Comp. | 5/1 | 0.167 | 180 | 15700 | 0.098 | 26% | 1.10 | 55 |
| Ex. 2 | | | | | | | | |
| Comp. | 5/1 | 0.167 | 180 | 11800 | 0.084 | 26% | 1.27 | 51 |
| Ex. 3 | | | | | | | | |
| Comp. | 2/1 | 0.333 | 180 | 17200 | 0.103 | 20% | 1.30 | 74 |
| Ex. 4 | | | | | | | | |
| Comp. | 7.5/1 | 0.118 | 180 | 20500 | 0.118 | 45% | 1.01 | 66 |
| Ex. 5 | | | | | | | | |
| Comp. | 10/1 | 0.091 | 180 | — | — | 52% | 0.79 | — |
| Ex. 6 | | | | | | | | |

The copolymers of Comparative Examples 1 to 6 were produced by conventional methods (reaction temperature: 180° C.), while the copolymers of Examples 1 to 10 were produced by special methods (for example, reaction temperature: 140 to 160° C., and other conditions such as stirring condition are controlled). As a result, in the copolymers of Examples 1 to 10, the amide bond proportion is higher as compared with the copolymers of Comparative Examples 1 to 5 having the same compositions, as apparent from Table 2 and FIG. 1. Accordingly, Tg is improved (heat resistance is improved) in the copolymers of the examples when copolymers having the same aspartic acid content and the same molecular weight are compared. The copolymers of Examples 1 to 10 are useful for a degradation promoting agent in which a carboxylic acid is effective for promotion of degradation since the copolymers have high acid value though Tg is not low.

### <Change of Tg by Change of Mw>

The change of Tg by the change of Mw during the polymerization reaction in Example 1 and Comparative Example 1 was measured. The results are shown in Table 3.

TABLE 3

| | |
|---|---|
| Comp. Ex. 1 | Ex. 1 |

| Mw | Tg | Mw | Tg |
|---|---|---|---|
| g/mol | ° C. | g/mol | ° C. |
| 12700 | 50 | 10600 | 52 |
| 13000 | 52 | 12700 | 58 |
| 17000 | 55 | 17900 | 62 |

As understood from Table 3, Tg in Example 1 is higher than Tg in Comparative Example 1 when copolymers having approximately the same molecular weight are compared. Such relatively high Tg is advantageous for performances such as preservation stability.

### <Solubility Test>

About 200 mg of the copolymers of Examples 1 to 10 were added into 10 mL of ion exchanged water, the mixture was stirred at room temperature for 1 hour, and solubility thereof in water was examined. All the copolymers were not dissolved at all. In contrast, a 0.1 mol/L sodium hydroxide aqueous solution was dropped onto about 5 g of the copolymer of Comparative Example 2, to cause ring-opening of a succinimide portion in the copolymer, referring to Patent Document 1. Then, the liquid was neutralized with 0.1 mol/L hydrochloric acid, and a chloroform/methanol solvent was added to cause deposition of sodium chloride which was then filtrated, and the filtrate was vacuum-dried and freeze-dried, to obtain a water-soluble compound in which a succinimide portion is ring-opened. Tg of this water-soluble compound was 47.2° C. Further, when solubility in water was examined, the degree of solubility was about 12% by mass. When the compound was left in air at room temperature, stickiness occurred, that is, the compound had very high hygroscopicity. As described in Patent Document 1, when an imide bond is converted to an amide bond by ring-opening, the amide bond proportion is supposed to increase, however, it changes to water-soluble, Tg lowers and hygroscopicity increases. In contrast, the copolymer of the present invention having an amide bond at a specific proportion already in polymerization is water-insoluble, has relatively high Tg and has low hygroscopicity, thus, is excellent in preservation stability.

### <High Temperature Preservation Stability Test>

Each 100 g of powders of the copolymer of Example 2 and the copolymer of Comparative Example 2 were sealed in aluminum bags, and stored in an oven of 50° C. for 1 month, then, taken out. The copolymer of Example 2 was loosened easily by hand after taking out, to show the original powdery state, while the copolymer obtained in Comparative Example 2 fused, to give a whole clump.

### <Hydrolysis Promoting Test>

Each 30 parts by mass of the copolymers of Examples 1 to 6 and Comparative Examples 1 to 5 and 70 parts by mass of polylactic acid (manufactured by NatureWorks, trade name: Ingeo 6302D) were kneaded for 10 minutes under conditions of 180° C. and 100 rpm using Micro Compounder manufactured by DSM, to obtain strands. In this kneading, a difference in lowering of the molecular weight was not recognized between the copolymers of Examples 1 to 6 and the copolymers of Comparative Examples 1 to 5. Next, the resultant strands were melted and pressed in vacuum to fabricate sheets having a thickness of about 160 μm, which were then cut into 20 mm square, to obtain test pieces.

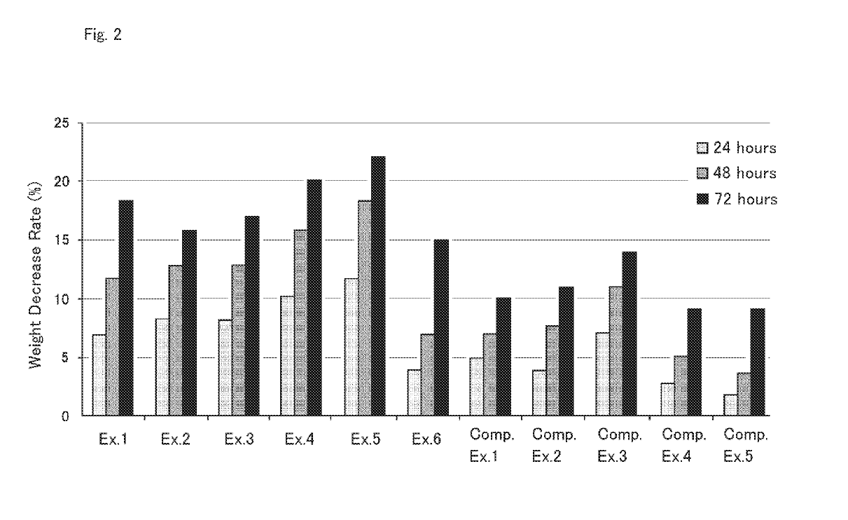

The precisely-weighed test piece (20×20 mm) and 8 mL of deionized water were added to a 20 cc sample tube and the tube was sealed, and the tube was allowed to stand still for prescribed time at a temperature of 60° C., and then, the sample tube was quenched. The resultant degraded liquid was filtrated through a paper filter (manufactured by Kiriyama Glass Works CO., trade name: Kiriyama filter paper No. 5C), and the resultant residue was washed with 10 mL of distilled water twice. The washed residue was dried under reduced pressure at room temperature under a trace amount of nitrogen flow until the weight became constant, and weighed, and the degradation rate was calculated as the reduction rate from the weight before the test. The results are shown in Table 4. Further, the results are graphed in FIG. 2.

TABLE 4

| | 30 parts by weight polylactic acid mixed sheet | | | |
|---|---|---|---|---|
| | 60° C., 24 h | 60° C., 48 h | 60° C., 72 h | 60° C., 96 h |
| | Degradation | Degradation | Degradation | Degradation |
| Polymer | rate % | rate % | rate % | rate % |
| Ex. 1 | 7 | 12 | 18 | 22 |
| Ex. 2 | 8 | 13 | 16 | — |
| Ex. 3 | 8 | 13 | 17 | 22 |
| Ex. 4 | 10 | 16 | 20 | — |
| Comp. Ex. 1 | 5 | 7 | 10 | — |
| Comp. Ex. 2 | 4 | 8 | 11 | 16 |
| Comp. Ex. 3 | 7 | 11 | 14 | — |

As apparent from Table 4 and FIG. 2, the compositions obtained by mixing the copolymers of Examples 1 to 6 having a lot of amide bonds and having high acid value showed higher weight decrease rate by hydrolysis as compared with the compositions obtained by mixing the copolymers of Comparative Examples 1 to 5 having a small number of amide bonds and having low acid value. It is believed that this is caused by improvement in compatibility by the increase in the amide bond proportion, and by promotion of degradation by an increase in the content of a carboxyl group having a catalytic action of hydrolysis.

Surprisingly, even when Example 6 (molar ratio of lactic acid to aspartic acid: 7.5/1, acid value: 1.12 mmol/g) having the lowest aspartic acid proportion among Examples 1 to 6 and Comparative Example 4 (molar ratio of lactic acid to aspartic acid: 2/1, acid value: 1.30 mmol/g) having the highest aspartic acid proportion among Comparative Examples 1 to 5 were compared, the weight decrease rate by hydrolysis was larger in Example 6 than in Comparative Example 4. It is understood from this fact that when a copolymer having an amide bond proportion in a specific range as in the present invention is used, excellent hydrolysis can be manifested even if the proportion of aspartic acid (amino group-containing polyvalent carboxylic acid) in the copolymer is low.

### INDUSTRIAL APPLICABILITY

The resin composition containing the copolymer (A) of the present invention and another resin is useful in various applications such as applications as vessel, film and fiber, or applications in the pharmaceutical field (sustained release medicine), as the biodegradable resin composition in which hydrolysis is promoted.

1. A water-insoluble copolymer having a constitutional unit (X) derived from a hydroxycarboxylic acid and a constitutional unit (Y) derived from an amino group-containing polyvalent carboxylic acid, wherein
the molar ratio (X/Y) of the constitutional unit (X) to the constitutional unit (Y) is 2/1≤(X/Y)<8/1, and
the amide bond proportion of the constitutional unit (Y) represented by the following formula (1) is defined by the following formulae (2-1) to (2-3):
amide bond proportion (%)=A/Asp×100 (1)
(wherein, A is the number of moles of an amide bond in the constitutional unit (Y) calculated by the ¹H-NMR spectrum measured in deuterated dimethylformamide, and Asp is the number of moles of the constitutional unit (Y) in the copolymer.)
[when 2/1≤(X/Y)<4/1]
amide bond proportion (%)≥25 (2-1)
[when 4/1≤(X/Y)≤6.5/1]
amide bond proportion (%)≥30 (2-2)
[when 6.5/1<(X/Y)<8/1]
amide bond proportion (%)≥50 (2-3).

2. The copolymer according to claim 1, wherein the weight-average molecular weight measured by size exclusion chromatography using dimethylacetamide as an eluent is 8000 or more and 50000 or less.

3. The copolymer according to claim 1, wherein the inherent viscosity in dimethylacetamide is 0.05 dl/g or more and 0.20 dl/g or less.

4. The copolymer according to claim 1, wherein the acid value is 0.2 mmol/g or more and 2.5 mmol/g or less.

5. The copolymer according to claim 1, wherein the copolymer has a glass transition temperature of 40° C. or higher and is amorphous having substantially no melting point.

6. A production method of the copolymer of claim 1, comprising a step of polymerizing a hydroxycarboxylic acid and an amino group-containing polyvalent carboxylic acid by direct dehydration and condensation.

7. The production method according to claim 6, wherein the polymerization is conducted at a reaction temperature of 170° C. or lower until the amino group-containing polyvalent carboxylic acid is dissolved.

8. The production method according to claim 6, wherein the polymerization is conducted at a reaction pressure of 100 mmHg or less.

9. The production method according to claim 6, wherein the polymerization is conducted using a catalyst.

10. The production method according to claim 9, wherein the polymerization is conducted using one or two or more kinds of catalysts selected from the group consisting of tin, titanium, zinc, aluminum, calcium, magnesium and organic acids.

11. A resin composition comprising the copolymer (A) of claim 1 and a resin (B) selected from the group consisting of polyolefin resins, polystyrene resins, polyester resins, polycarbonate resins and degradable resins, wherein the mass ratio (A/B) of the copolymer (A) to the resin (B) is 1/99 to 50/50.

12. The resin composition according to claim 11, wherein the resin (B) is a degradable resin.

13. The resin composition according to claim 12, wherein the degradable resin is an aliphatic polyester.

14. The resin composition according to claim 11, wherein the reduced viscosity of the copolymer (A) in dimethylacetamide is 0.05 or more and 0.20 or less.

15. A method for promoting hydrolysis of a resin (B) having a weight-average molecular weight of 3000 or more and 500000 or less selected from the group consisting of polyolefin resins, polystyrene resins, polyester resins, polycarbonate resins and degradable resins, wherein the copolymer (A) according to claim 1 is mixed with the resin (B) so that the mass ratio (A/B) of the copolymer (A) to the resin (B) is 1/99 to 50/50.

16. The method according to claim 15, wherein the resin (B) is an aliphatic polyester.
